(19) United States
(12) Patent Application Publication
(10) Pub. No.: US 2013/0149381 A1
(43) Pub. Date: Jun. 13, 2013

(54) ABSORPTION METHOD FOR ENTRAPMENT OF DRUGS IN POLYMERIC NANOPARTICLES

(71) Applicant: Centro De Investigacion En Quimica Aplicada, Saltillo (MX)

(72) Inventors: Raúl Guillermo López Campos, Saltillo (MX); Hened Saade Caballero, Saltillo (MX)

(73) Assignee: CENTRO DE INVESTIGACION EN QUIMICA APLICADA, Saltillo (MX)

(21) Appl. No.: 13/708,825

(22) Filed: Dec. 7, 2012

Related U.S. Application Data
(60) Provisional application No. 61/568,567, filed on Dec. 8, 2011.

(51) Int. Cl. A61K 9/51

(52) U.S. Cl. CPC A61K 9/5192; Y10S 977/773; Y10S 977/906; Y10S 977/897; B82Y 5/00
USPC 424/489; 514/570; 977/773; 977/906; 977/897

(57) ABSTRACT

A method for preparing polymeric nanoparticles having entrapped active ingredients or drugs, the method includes the step of preparing a reaction by mixturing water, a surfactant, and a water-soluble radical initiator; polymerizing a polymerizable monomer in the reaction to obtain a dispersion of polymeric nanoparticles having a controlled size with average diameters smaller than 50 nm; dissolving one or more active ingredients in a suitable solvent; adding the solution of active ingredients to the dispersion of polymeric nanoparticles and allowing that the active ingredients to become entrapped within polymeric nanoparticles; and evaporating the dispersion of polymeric nanoparticles having entrapped active ingredients to evaporate the residual monomer and the solvent used as a vehicle for loading the active ingredient.

### TECHNICAL FIELD OF THE INVENTION

This invention relates to a method for producing nanoparticles, more particulary a method for preparing polymeric nanoparticles having entrapped active ingredients.

### BACKGROUND OF THE INVENTION

Entrapment of drugs in polymeric nanoparticles is an approach for drugs delivery, which offers some interesting advantages over more conventional dosage methods. Among these advantages, it can be mentioned an increase in the drug targeting to the site of action and a reduction of side effects. These advantages mainly arise from the very small size of the nanoparticles. Different studies have shown that reduction in particle size (diameters<30 nm) makes more difficult to reticulo-endothelial system to recognize them, increasing their half-life in the blood stream. Polymeric nanoparticles with dissolved, entrapped, encapsulated or attached drugs are prepared by a number of methods, which make use of preformed polymers or polymers synthesized during the process. These methods allow obtaining nanoparticles as small as 50-100 nm in average diameter; however, they are not able to prepare smaller particles. This plurality of techniques is due in part to the fact that no current technique provides a reliable, simple, and low-cost method for production of nanoparticles of a controlled size. Other techniques suffer from an inability to control the distribution of sizes around a desired nanoparticle size. Still other nanoparticle synthesis techniques require specialized equipment, long processing times, or expensive specialty chemicals,

The reported methods for preparing drugs containing polymeric nanoparticles based on the use of preformed polymers, usually include the preparation of a drug solution, which is dispersed as nanodroplets in a continuous phase by using surfactants and a combination of high-speed homogenization and sonication. The polymer can be dissolved either in the dispersed or the continuous phase. Some of the methods using this approach are i) nanoprecipitation; ii) emulsion-diffusion; iii) double emulsification; iv) emulsion-coacervation; v) polymer-coating; vi) layer-by-layer; and, vii) solvent evaporation. The reason why very tiny particles cannot be obtained by using these methods, is the lower limit to droplet size imposed by the use of homogenization and sonication.

### SUMMARY OF THE INVENTION

The object of the invention is to provide a method for preparing polymeric nanoparticles having entrapped active ingredients, the method includes the step of preparing an aqueous phase for carry out a polymerization reaction by mixturing water, a surfactant, a water-soluble radical initiator, and, optionally, a chain transfer agent; polymerizing a polymerizable monomer in the aqueous phase to obtain a dispersion of polymeric nanoparticles having a controlled size with average diameters smaller than 50 nm; dissolving one or more active ingredients in a suitable solvent; adding the solution of active ingredients to the dispersion of polymeric nanoparticles and allowing to one or more active ingredients to become entrapped within polymeric nanoparticles; and evaporating under reduced pressure said dispersion of polymeric nanoparticles having entrapped active ingredients to evaporate the residual monomer and the solvent used as a vehicle for loading the active ingredient.

### BRIEF DESCRIPTION OF THE FIGURES

The characteristic details of the invention are described in the following paragraphs together with the attached drawings, with the purpose of defining the invention, but without limiting its range.

FIG. 1 shows a micrograph determined by transmission electronic microscopy (TEM) of a sample of poly(methyl methacrylate)-Ibuprofen (PMMA-IB) nanoparticles of latex (a) and a histogram of the particle diameter distribution (b) according to the invention.

FIG. 2 shows a micrograph determined by TEM of a sample of PMMA-IB nanoparticles of latex (a) and a histogram of the particle diameter distribution (b) according to the invention.

### DETAILED DESCRIPTION OF THE INVENTION

The following description is only intended to represent the way of how the principles of the invention can be implemented in various embodiments. The embodiments described herein do not intend to be a comprehensive representation of the invention. The following embodiments are not to limit the invention to the precise form published in the following detailed description.

The terms “active ingredient” or “drug” as used herein refer to a medicinal material, a compound or a mixture thereof, suitable and medically indicated for treatment of a malcondition in a patient. The agent can be in a solid physical form or a liquid physical form at about room temperature or at about body temperature, depending on the melting point of the material.

Preparation of Polymeric Nanoparticles A glass reactor is loaded, jacketed, fitted with mechanical stirring and a reflux system with the necessary amounts of water, a surfactant, and a water-soluble radical initiator, and chain transfer agent, if required, and the system is sealed to prevent the entrance of air. Argon is then bubbled into the mixture for 1 h. Then, 10 min before starting the monomer dosage, the water recirculating bath is connected through the jacket of the reactor to reach the reaction temperature while maintaining a stirring rate of 650 rpm. The polymerization started with the initiation of dosing a polymerizable monomer to a constant, previous predetermined flow by a dosing pump. The latex obtained is poured into a suitable container from which a sample is taken to determine the final conversion by gravimetry. To perform the loading of the drug, a portion of approximately 50 g latex is taken and diluted with water in a 1:1 weight ratio.

The size and stability of the nanoparticles is dependent upon the amount of water loading and the molar ratio of water to surfactant. The surfactant used to disperse the monomer may be nonionic, anionic or cationic in kind. The surfactants may be anionic: for example, sodium dodecylsulfate (SDS), salts of fatty acids, such as salts of dialkylsulfosuccinic acid, especially sodium dioctyl sulfosuccinate (AOT), salts of alkyl and aryl sulfonates and salts of tri-chain amphiphilic compounds, such as sodium trialkyl sulfo-tricarballylates. The anionic surfactants may also comprise hydrophilic non-ionic functionalities, such as ethylene oxide or hydroxyl groups. They may be nonionic: for example, polyoxyethylene alkyl ethers, acetylene diols and their derivatives, alkylthiopolyacrylamides, copolymers of polyoxyethylene and polyoxypropylene, alcohol alkoxylates, sugar-based derivatives; they may be cationic, such as alkyl amines, quaternary ammonium salts; or they may be amphoteric: for example, betaines. However the surfactant should normally be selected such that it is either uncharged (non-ionic), has no overall charge (amphoteric or zwitterionic surfactant) or matches the charge of the stimulus-responsive polymer used. The preferred are SDS and AOT. The surfactants may be incorporated in the initial reaction mixture in an amount of 0.1% to 15%, preferably 0.5% to 12.5%, in particular 1% to 10%, by weight based on the total weight of the aqueous phase.

The polymerization may be initiated using a charged or chargeable water-soluble radical initiator species, such as, for example, a salt of the persulfate anion, especially potassium persulfate, or with a neutral initiator species if a charged or chargeable co-monomer species is incorporated in the preparation. The initiation of the radical polymerization may then triggered by the decomposition of the initiatior resulting from exposure to heat or to light. In the case of initiation using heat, a reduced temperature can be used by combining the initiator compound, such as potassium persulfate, with an accelerator compound, such as sodium metabisulfite. The water-soluble radical initiator is used preferably in an amount of 0.01 to 10%, preferably 0.05% to 7.5%, in particular 0.1% to 5%, by weight based on the total weight of the monomer.

In an alternative embodiment of the invention, a chain transfer agent may be added to the reaction. The chain transfer agent is a molecule which is known to reduce molecular weight during a free-radical polymerization via a chain transfer mechanism. These agents may be any thiol-containing molecule and can be either monofunctional or multifunctional. The agent may be hydrophilic, hydrophobic, amphiphilic, anionic, cationic, neutral, zwitterionic or responsive. The molecule can also be an oligomer or a pre-formed polymer containing a thiol moiety. (The agent may also be a hindered alcohol or similar free-radical stabiliser). Catalytic chain transfer agents such as those based on transition metal complexes such as cobalt bis(borondifluorodimethyl-glyoximate) (CoBF) may also be used. Suitable thiols include but are not limited to C2-C18 alkyl thiols such as dodecane thiol, thioglycolic acid, thioglycerol, cysteine and cysteamine. Thiol-containing oligomers or polymers may also be used such as poly(cysteine) or an oligomer or polymer which has been post-functionalised to give a thiol group(s), such as polyethyleneglycol) (di)thio glycollate, or a pre-formed polymer functionalised with a thiol group, for example, reaction of an end or side-functionalised alcohol such as poly(propylene glycol) with thiobutyrolactone, to give the corresponding thiol-functionalised chain-extended polymer. Multifunctional thiols may also be prepared by the reduction of a xanthate, dithioester or trithiocarbonate end-functionalised polymer prepared via a Reversible Addition Fragmentation Transfer (RAFT) or Macromolecular Design by the Interchange of Xanthates (MADIX) living radical method. Xanthates, dithioesters, and dithiocarbonates may also be used, such as cumyl phenyldithioacetate. Alternative chain transfer agents may be any species known to limit the molecular weight in a free-radical addition polymerization including alkyl halides and transition metal salts or complexes. More than one chain transfer agent may be used in combination. When the chain transfer agent is providing the necessary hydrophilicity in the copolymer, it is preferred that the chain transfer agent is hydrophilic and has a molecular weight of at least 1000 Daltons.

Hydrophilic chain transfer agents typically contain hydrogen bonding and/or permanent or transient charges. Hydrophilic chain transfer agents include but are not limited to thio-acids such as thioglycolic acid and cysteine, thioamines such as cysteamine and thio-alcohols such as 2-mercaptoethanol, thioglycerol and ethylene glycol mono- (and di-)thio glycollate. Hydrophilic macro-CTAs (where the molecular weight of the CTA is at least 1000 Daltons) can be prepared from hydrophilic polymers synthesised by RAFT (or MADIX) followed by reduction of the chain end, or alternatively the terminal hydroxyl group of a preformed hydrophilic polymer can he post functionalised with a compound such as thiobutyrolactone.

Hydrophobic chain transfer agents include but are not limited to linear and branched alkyl and aryl (di)thiols such as dodecanethiol, octadecyl mercaptan, 2-methyl-1-butanethiol and 1,9-nonanedithiol.

The chain transfer agent is used preferably in an amount of 0.01 to 10%, preferably 0.05% to 7.5%, in particular 0.1% to 5%, by weight based on the total weight of the monomer.

Specific examples of the polymerizable monomers for use in preparing the nanoparticles of the invention include mono- or poly-functional vinyl monomers which can be radically polymerized.

Specific examples of the monofunctional polymerizable monomers include styrene derivatives such as styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene, and p-phenylstyrene; acrylic monomers such as methyl acrylate, ethyl acrylate, n-propyl acrylate, iso-propyl acrylate, n-butyl acrylate, iso-butyl acrylate, tert-butyl acryalte, n-amyl acrylate, n-hexyl acrylate, 2-ethylhexyl acryalte, n-octyl acryalte, n-nonyl acrylate, cyclohexyl acrylate, benzyl acryalte, dimethylphosphate ethyl acylate, diethylphosphate ethyl acylate, dibutylphosphate ethyl acylate, and 2-benzoyloxyethyl acrylate; methacrylic monomers such as methyl methacrylate, ethyl methacrylate, n-propyl methacrylate, iso-propyl methacrylate, n-butyl methacrylate, iso-butyl methacrylate, tert-butyl methacryalte, n-amyl methacrylate, n-hexyl methacrylate, 2-ethylhexyl methacryalte, n-octyl methacryalte, n-nonyl methacrylate, diethylphosphate ethyl methacylate, dibutylphosphate ethyl methacylate; vinyl esters such as methylenealiphaticmonocarboxylic acid esters, vinyl acetate, vinyl propionate, vinyl butyrate, vinyl benzoate, vinyl formate; vinyl ethers such as vinyl methyl ether, vinyl ethyl ether, and vinyl isobutyl ether; vinyl ketones such as vinyl methyl ketone, vinyl hexyl ketone, vinyl isopropyl ketone, and combinations thereof.

Specific examples of the polyfunctional polymerizable monomers include diethylene glycol diacryalte, triethylene glycol diacrylate, tetraethylene glycol diacrylate, polyethylene glycol diacrylate, 1,6-hexanediol diacryalte, neopentyl glycol diacrylate, tripropylene glycol diacrylate, polypropylene glycol diacrylate, 2,2′-bis{4-(acryloxydiethoxy)phenyl}propane, trimethylolpropane triacrylate, tetramethylolmethane tetraacrylate, ethylene glycol dimethacrylate, diethylene glycol dimethacrylate, triethylene glycol dimethacrylate, tetraethylene glycol dimethacrylate, polyethylene glycol dimethacrylate, 1,3-butylene glycol dimethacrylate, 1,6-hexanediol dimethacryalte, neopentyl glycol dimethacrylate, polypropylene glycol dimethacrylate, 2,2′-bis{4-(methacryloxydiethoxy)phenyl}propane, 2,2′-bis{4-(methacryloxypolyethoxy)phenyl}propane, trimethylolpropane trimethacrylate, tetramethylolmethane tetramethacrylate, divinyl ether, and combinations thereof.

The monofunctional polymerizable monomers mentioned above can be used alone or in combination. In addition, polyfunctional polymerizable monomers can be used together with one or more of monofunctional monomers. Among the monomers mentioned above, styrene, (meth)acrylic acid and/or their derivatives are preferably used alone or in combination with other monomers.

The preferred polymerizable monomer is methyl methacrylate. The polymerizable monomer may be incorporated in the mixture in an amount of 0.1 to 50%, preferably 0.5% to 40%, in particular 5% to 30%, by weight based on the total weight of the reaction mixture.

This polymerization technique allows obtaining polymeric nanoparticles with average diameters smaller than 50 nm, usually between 10 and 40 nm, dispersed in a continuous phase.

### Loading of the Drug in the Polymeric Nanoparticles

Active ingredients or drugs to be entrapped in the polymers nanoparticles, include but are not limited to, drug molecules, peptides/proteins, imaging agents, genetic material or a combination of these compounds.

The active ingredients or drugs can be organic compounds that are poorly soluble or insoluble in water but readily soluble in organic solvents. The active ingredients or drugs agent is added to the polymeric dispersion either in the form of dry powder or as a solution in dichloromethane, chloroform, ethanol or ether depending on the solubility of the drug in that solvent to form an optically clear solution. Examples of such active ingredients o drugs include, but are not limited to, antineoplastic agents such as Paclitaxel, Docetaxel, Rapamycin, Doxorubicin, Daunorubicin, Idarubicin, Epirubicin, Capecitabine, Mitomycin C, Amsacrine, Busulfan, Tretinoin, Etoposide, Chlorambucil, Chlormethine, Melphalan, and Benzylphenylurea (BPU) compounds; phytochemicals and other natural compounds such as curcumin, curcuminoids, and other flavinoids; steroidal compounds such as natural and synthetic steroids, and steroid derivatives like cyclopamine; antiviral agents such as Aciclovir, Indinavir, Lamivudine, Stavudine, Nevirapine, Ritonavir, Ganciclovir, Saquinavir, Lopinavir, Nelfinavir; antifungal agents such as Itraconazole, Ketoconazole, Miconazole, Oxiconazole, Sertaconazole, Amphotericin B, and Griseofulvin; antibacterial agents such as quinolones including Ciprofloxacin, Ofloxacin, Moxifloxacin, Methoxyfloxacin, Pefloxacin, Norfloxacin, Sparfloxacin, Temafloxacin, Levofloxacin, Lomefloxacin, Cinoxacin; antibacterial agents such as penicillins including Cloxacillin, Benzylpenicillin, Phenylmethoxypenicillin; antibacterial agents such as aminoglycosides including Erythromycin and other macrolides; antitubercular agents such as rifampicin and rifapentin; and anti-inflammatory agents such as Ibuprofen, Indomethacin, Ketoprofen, Naproxen, Oxaprozin, Piroxicam, Sulindac. Preferably, the active ingredients or drugs loaded in the nanoparticles is in a range from 1% to 50% by weight of the polymer; however, in some applications the loading may be considerably higher.

To carry out the active ingredient loading in the polymer nanoparticles, first a solution of the drug is prepared in a suitable solvent to a predetermined concentration. The suitable solvent in the present invention is a halogenated hydrocarbon or alkyl halide having 1 to 4 carbon(s) and, to be more specific, it is dichloromethane, chloroform, carbon tetrachloride, dichloroethane, 2,2,2-trichloroethane or the like. Preferably, dichloromethane is used. The solvent may be incorporated in the reaction mixture in an amount of 0.1 to 50%, preferably 0.5% to 20%, in particular 1% to 10%, by weight based on the total weight of the polymer dispersion.

The prediluted latex is loaded in the same reactor where the polymerization was carried out. It was taken to the temperature at which the loading of the drug-solvent solution would be carried out, maintaining the stirring rate at 650 rpm. Next, dosing began of the drug-solvent solution with discontinuous aggregates. This involved the addition of a load of known weight between 0.4 and 0.6 g, leaving sufficient time until the appearance of the dispersion is again translucent, as it is at the start of the test, before proceeding with the next aggregate. Once the dosage of the drug-solvent solution is finished, and given the time necessary for the dispersion to resume its translucent appearance, said solution is poured into a rotary evaporator to evaporate under reduced pressure the residual monomer and the solvent used as a vehicle for loading the active ingredient (i.e. Ibuprofen). Thereafter samples are taken to determine solids content and particle size by quasi-elastic light scattering (QLS) and transmission electronic microscopy (TEM).

The nanoparticles containing at least one active ingredient or drug or a combination of active ingredients or drugs prepared by the above described process (e.g., nanoparticles with entrapped active ingredients or drugs) may be used for the treatment of pathological conditions arising out of various diseases including but not limited to cancer, inflammation, infection and neurodegeneration.

A benefit of the present invention is the ability to generate polimeric nanoparticles of controlled size and composition, where the size of particles in a population can be substantially homogeneous. The polymeric nanoparticles of the present invention comprise an entraped substantially pure active ingredient or drug. It will be understood by one of skill in the art that two or more active ingredients can be co-formulated, in which case the purity shad refer to the combined active agents.

The polymeric nanoparticles has a controlled size. Usually this is at least about 5 nm in diameter, more usually at least about 35 nm in diameter, at least about 50 nm in diameter. The polymeric nanoparticles may have a defined size range, which may be substantially homogeneous, where the variability may be not more than 100% of the diameter, not more 50%, not more than 10%.

### EXAMPLES OF THE INVENTION

The invention will now be described with respect to the following examples, which are solely for the purpose of representing the way of carrying out the implementation of the principles of the invention. The following examples are not intended to be a comprehensive representation of the invention, or try to limit the scope thereof.

### Example 1

The reactor was loaded with 93.1358 g of water, 3.6741 g of the sodium dodecyl sulfate surfactant, 1.2247 g of bis (2-ethylhexyl sodium sulfosuccinate) and 0.0413 g of potassium persulfate initiator. Sealing the reaction system was activated at 650 rpm stirring and argon was bubbled into the mixture for 1 h. Water started to pass through the reactor jacket to achieve the reaction temperature of 60° C., maintaining the stirring rate at 650 rpm. Dosing was initiated of the monomer methyl methacrylate to a flow of 0.07 g/min. Dosing is continued for 6 hours, adding a total of 24.9321 g of monomer methyl methacrylate. The latex obtained was poured into a suitable container from which a sample was taken to determine the final conversion by gravimetry, which was 96.8%. From the latex 45.9072 g were taken, which were diluted with 46.0100 g of water.

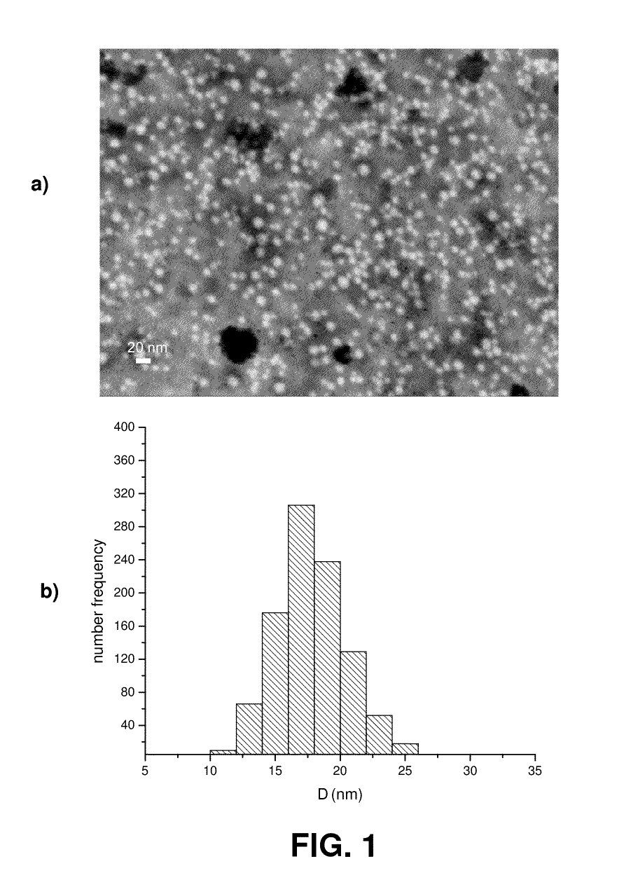

For the loading of Ibuprofen, first a solution of the drug in dichloromethane was prepared at 39.8% at 30° C. The diluted latex was loaded into the reactor used for polymerization, it was sealed, agitation was activated at 650 rpm and the mixture was heated to 30° C. Thereafter, the Ibuprofen-Dichloromethane solution started to add to a total of 5.7484 g. The aggregate total time, including the time since the last aggregate to the dispersion resumed its original translucency, was 330 min. From the final dispersion, a sample was taken and analyzed to determine its solids content. Afterwards, the dispersion was filtered through a filter of 0.2 μm, a sample taken of the filtered substance, and analyzed to determine the solids content. The solids content before and after filtration were 13.5 and 13.7%, respectively, this is, very similar to each other. This indicated that there were no aggregates of Ibuprofen larger than 200 nm outside the poly(methyl methacrylate) nanoparticles. The content of Ibuprofen in dried nanoparticles determined by UV spectrophotometry was 18.9%. According to the formulation used, a content of 17.4% was expected. The poly(methyl methacrylate)-Ibuprofen particle average diameters determined by QLS and TEM were both 17.8 nm. The QLS measuring gave only one particle population, which would correspond to PMMA-Ibuprofen nanoparticles with 17.8 nm in mean diameter. This discards the presence of Ibuprofen aggregates equal or smaller than 200 nm in size, demonstrating that all the added Ibuprofen was loaded in the nanoparticles. FIG. 1 shows a representative micrograph of the PMMA-Ibuprofen nanoparticles (a) and the histogram of particle size (b).

### Example 2

The reactor was loaded with 93.1026 g of water, 3.6802 g of the sodium dodecyl sulfate surfactant, 1.2268 g of bis (2-ethylhexyl sodium sulfosuccinate) and 0.044 g of potassium persulfate initiator. Sealing the reaction system was activated at 650 rpm stirring and argon was bubbled into the mixture for 1 h. Water started to pass through the reactor jacket to achieve the reaction temperature of 60° C., maintaining the stirring rate at 650 rpm. Dosing was initiated of the monomer methyl methacrylate to a flow of 0.07 g/min. Dosing is continued for 6 hours, adding a total of 25.4481 g of monomer methyl methacrylate. The latex obtained was poured into a suitable container from which a sample was taken to determine the final conversion by gravimetry, which was 93.1%. From the latex 54.8984 g were taken, which were diluted with 54.6115 g of water.

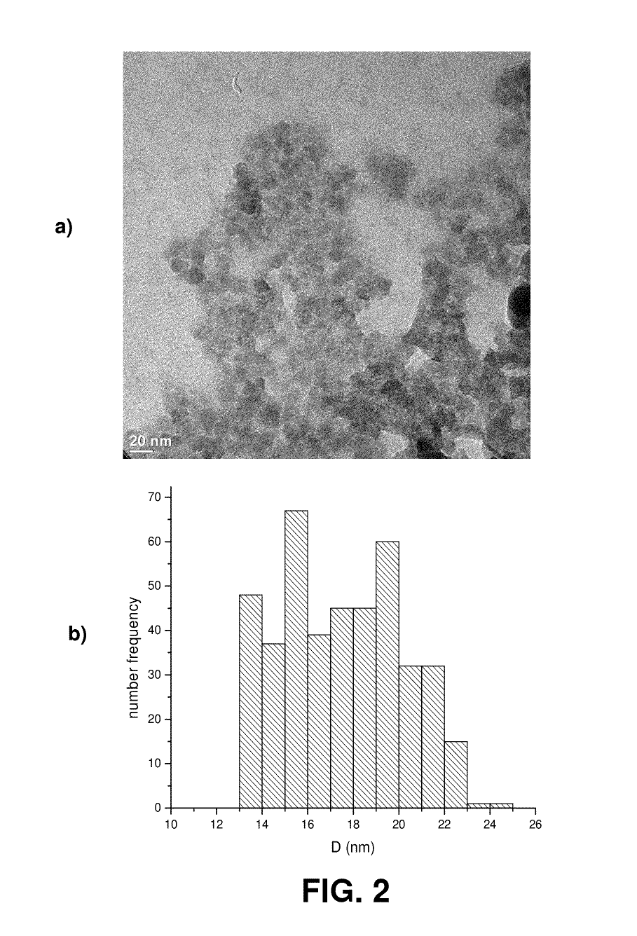

For the loading of Ibuprofen, first a solution of the drug in dichloromethane was prepared at 40.0% at 30° C. The diluted latex was loaded into the reactor used for polymerization, it was sealed, agitation was activated at 650 rpm and the mixture was heated to 60° C. Thereafter, the Ibuprofen-Dichloromethane solution started to add to a total of 6.4789 g. The aggregate total time, including the time since the last aggregate to the dispersion resumed its original translucency, was 453 min. From the final dispersion, a sample was taken and analyzed to determine its solids content. Afterwards, the dispersion was filtered through a filter of 0.2 μm, a sample taken of the filtered substance, and analyzed to determine the solids content. The solids content before and after filtration were 14.15 and 14.33%, respectively, this is, very similar to each other. This indicated that there were no aggregates of Ibuprofen larger than 200 nm outside the poly(methyl methacrylate) nanoparticles. The content of Ibuprofen in dried nanoparticles determined by UV spectrophotometry was 19.1%. According to the formulation used, a content of 16.9% was expected. The poly(methyl methacrylate)-Ibuprofen particle average diameters determined by QLS and TEM were 18.2 and 17.4 nm, respectively (see FIG. 2). The QLS measuring gave only one particle population, which would correspond to PMMA-Ibuprofen nanoparticles with 18.2 nm in mean diameter. This discards the presence of Ibuprofen aggregates equal or smaller than 200 nm in size, demonstrating that all the added Ibuprofen was loaded in the nanoparticles. FIG. 2 shows a representative micrograph of the PMMA-Ibuprofen nanoparticles (a) and the histogram of particle size (b).

Although the invention was described with reference to specific embodiments, this description is not intended to be built in a limited sense. The different modifications of the embodiments published, as well as alternative embodiments of the invention will be apparent to persons knowledgeable in the state of the art when referring to the description of the invention. For this reason it is considered that the appended claims cover such modifications that fall within the scope of the invention, or their equivalents.

1. A method for preparing polymeric nanoparticles having entrapped active ingredients comprising the steps of:
preparing an aqueous phase for carry out a polymerization reaction by mixturing water, a surfactant, a water-soluble radical initiator, and, optionally, a chain transfer agent;
polymerizing a polymerizable monomer in said aqueous phase to obtain a dispersion of polymeric nanoparticles, wherein said polymeric nanoparticles have a controlled size with average diameters smaller than 50 nm;
dissolving one or more active ingredients in a suitable solvent;
adding said solution of active ingredients to said dispersion of polymeric nanoparticles and allowing said one or more active ingredients to become entrapped within polymeric nanoparticles; and
evaporating under reduced pressure said dispersion of polymeric nanoparticles having entrapped active ingredients to evaporate the residual monomer and the solvent used as a vehicle for loading the active ingredient.

2. The method of claim 1, wherein said aqueous phase includes:
from 85% to 99% by weight of said water;
from 0.1% to 15% by weight of said surfactant;
from 0.01% to 10% by weight of said water-soluble radical initiator based on the total weight of the monomer; and
from 0.01% to 10% by weight of said chain transfer agent based on the total weight of the monomer.

3. The method of claim 1, wherein said surfactant is selected form a group consisting of sodium dodecylsulfate (SDS), dialkylsulfosuccinic acid, sodium dioctyl sulfosuccinate (AOT), salts of alkyl, aryl sulfonates, salts of tri-chain amphiphilic compounds, sodium trialkyl sulfo-tricarballylates, ethylene oxide, hydroxyl groups, polyoxyethylene alkyl ethers, acetylene diols, alkylthiopolyacrylamides, copolymers of polyoxyethylene and polyoxypropylene, alcohol alkoxylates, sugar-based derivatives, alkyl amines, quaternary ammonium salts, betaines, and combination thereof.

4. The method of claim 3, wherein said surfactant is sodium dodecylsulfate (SDS), sodium dioctyl sulfosuccinate (AOT), and combinations thereof.

5. The method of claim 1, wherein said water-soluble radical initiator is a salt of persulfate anion.

6. The method of claim 5, wherein said salt of persulfate anion is potassium persulfate.

7. The method of claim 1, wherein said chain transfer agent selected from a group consisting of dodecanethiol, octadecyl mercaptan, 2-methyl-1-butanethiol and 1,9-nonanedithiol, and combinations thereof.

8. The method of claim 1, wherein said polymerizable monomer is in amount from 0.1% to 50% by weight based on the total weight of the reaction mixture.

9. The method of claim 1, wherein said polymerizable monomer is selected from a group consisting of styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene, p-phenylstyrene, methyl acrylate, ethyl acrylate, n-propyl acrylate, iso-propyl acrylate, n-butyl acrylate, iso-butyl acrylate, tert-butyl acryalte, n-amyl acrylate, n-hexyl acrylate, 2-ethylhexyl acryalte, n-octyl acryalte, n-nonyl acrylate, cyclohexyl acrylate, benzyl acryalte, dimethylphosphate ethyl acylate, diethylphosphate ethyl acylate, dibutylphosphate ethyl acylate, and 2-benzoyloxyethyl acrylate, methyl methacrylate, ethyl methacrylate, n-propyl methacrylate, iso-propyl methacrylate, n-butyl methacrylate, iso-butyl methacrylate, tert-butyl methacryalte, n-amyl methacrylate, n-hexyl methacrylate, 2-ethylhexyl methacryalte, n-octyl methacryalte, n-nonyl methacrylate, diethylphosphate ethyl methacylate, dibutylphosphate ethyl methacylate, methylenealiphaticmonocarboxylic acid esters, vinyl acetate, vinyl propionate, vinyl butyrate, vinyl benzoate, vinyl formate, vinyl methyl ether, vinyl ethyl ether, and vinyl isobutyl ether, vinyl methyl ketone, vinyl hexyl ketone, vinyl isopropyl ketone, and combinations thereof.

10. The method of claim 9, wherein said polymerizable monomer is methyl methacrylate.

11. The method of claim 1, wherein said active ingredient is in amount from 0.1% to 50% by weight based on the total weight of the reaction mixture.

12. The method of claim 1, wherein said active ingredient is selected from a group consisting of Paclitaxel, Docetaxel, Rapamycin, Doxorubicin, Daunorubicin, Idarubicin, Epirubicin, Capecitabine, Mitomycin C, Amsacrine, Busulfan, Tretinoin, Etoposide, Chlorambucil, Chlormethine, Melphalan, Benzylphenylurea (BPU) compounds, curcumin, curcuminoids, flavinoids, natural and synthetic steroids, cyclopamine, Aciclovir, Indinavir, Lamivudine, Stavudine, Nevirapine, Ritonavir, Ganciclovir, Saquinavir, Lopinavir, Nelfinavir, Itraconazole, Ketoconazole, Miconazole, Oxiconazole, Sertaconazole, Amphotericin B, Griseofulvin, quinolones, Ciprofloxacin, Ofloxacin, Moxifloxacin, Methoxyfloxacin, Pefloxacin, Norfloxacin, Sparfloxacin, Temafloxacin, Levofloxacin, Lomefloxacin, Cinoxacin, penicillins, Cloxacillin, Benzylpenicillin, Phenylmethoxypenicillin, aminoglycosides, Erythromycin, macrolides, rifampicin, rifapentin, Ibuprofen, Indomethacin, Ketoprofen, Naproxen, Oxaprozin, Piroxicam, Sulindac, and combinations thereof.

13. The method of claim 1, wherein said active ingredient is Ibuprofen.

14. The method of claim 1, wherein said active ingredient is in amount from 0.1% to 60% by weight based on the total weight of the solution suitable solvent-active ingredient.

15. The method of claim 1, wherein said suitable solvent is selected from a group consisting of dichloromethane, chloroform, carbon tetrachloride, dichloroethane, 2,2,2-trichloroethane, and combinations thereof.

16. The method of claim 15, wherein said suitable solvent is dichloromethane.

17. The method of claim 1, wherein said polymeric nanoparticles have an average diameter smaller from 10 nm to 40 nm.
